(19) United States
(12) Patent Application Publication
(10) Pub. No.: US 2013/0245710 A1
(43) Pub. Date: Sep. 19, 2013

(54) INTEGRAL STIMULATION LEAD

(71) Applicant: CARDIAC PACEMAKERS, INC., St. Paul, MN (US)

(72) Inventors: Arthur Foster, Blaine, MN (US); Matthew John Tracy, St Michael, MN (US); Howard D. Simms, JR., Shoreview, MN (US)

(73) Assignee: Cardiac Pacemakers, Inc., St. Paul, MN (US)

(21) Appl. No.: 13/766,232

(22) Filed: Feb. 13, 2013

Related U.S. Application Data
(60) Provisional application No. 61/612,821, filed on Mar. 19, 2012.

(51) Int. Cl. A61N 1/375

(52) U.S. Cl. CPC A61N 1/3752
USPC 607/37; 29/876

(57) ABSTRACT

An apparatus, such as included as a portion of cardiac rhythm management (CRM) system, can include a subcutaneously-implantable medical device. The subcutaneously-implantable medical device can include a header assembly including a stylet access port, and the apparatus can include an intravascularly-deliverable lead assembly permanently attached to the header assembly and including a lumen to receive a stylet via the stylet access port included in the header assembly.

### CLAIM OF PRIORITY

This application claims the benefit of priority under 35 U.S.C. §119(e) of Foster et al., U.S. Provisional Patent Application Ser. No. 61/612,821, titled “INTEGRAL STIMULATION LEAD,” filed on Mar. 19, 2012, which is hereby incorporated by reference herein in its entirety.

### BACKGROUND

The heart produces rhythmic contractions, and when functioning normally, pumps blood throughout the body to meet metabolic demand. The heart includes specialized conduction pathways that conduct impulses initiated from the sino-atrial (SA) node throughout the myocardium to elicit such rhythmic contractions. These specialized conduction pathways normally conduct depolarizations from the SA node to the atrial myocardium, to the atrio-ventricular node, and to the ventricular myocardium to produce a coordinated contraction of the heart. Various diseases or physiologic conditions can disturb such regular and coordinated rhythmic contractions. Such diseases can include various arrhythmias, such as bradyarrhythmias where the heart may beat too slowly, or tachyarrhythmias where the heart may beat too rapidly or in an uncoordinated manner. Treatment of such arrhythmias, or one or more other diseases such as congestive heart failure, can include using an implantable cardiac rhythm management (CRM) system.

Generally, a CRM system can provide electrostimulation to one or more heart chambers to elicit a contraction, to terminate an arrhythmia, or to provide coordination of contraction between different locations of the heart. Such a CRM system generally includes an implantable pulse generator assembly that can be coupled to one or more leads. The one or more leads are generally delivered via an intravascular route to a desired location inside the body of a patient, such as within the heart, and the one or more leads are then connected by the implanting caregiver to a lead connector included as a portion of an implantable pulse generator assembly. The pulse generator assembly can then be implanted subcutaneously or in a sub-muscular location.

U.S Patent Application Publication No. US 2009/0198295 A1, Dennis et al., discusses an implantable medical device that is configured so that all major components, including a housing and attached leads, are disposed within the vasculature of a patient.

### OVERVIEW

In generally-available cardiac rhythm management (CRM) systems, an interconnection between an implantable lead assembly and an implantable pulse generator can be made via a connector “header” coupled to a housing of the implantable pulse generator. Such a header can include one or more set-screws, such as coupled to one or more set-screw-blocks, and one or more set-screw access ports configured to permit an implanting caregiver, such as a physician, to electrically and mechanically couple one or more implantable leads to circuitry included as a portion of the implantable pulse generator during implantation.

The present inventors have recognized, among other things, that such user-attachable lead couplings between the one or more leads and the header can permit errors or can result in other difficulties. For example, a user may insert a lead assembly into an incorrect port or position in the header, or a user may under- or over-tighten a set screw included in the header, which can compromise an electrical or mechanical connection between the lead and the header. A user may also damage a seal between the set screw and medium surrounding the header during attachment of a lead. Such errors or difficulties can result in additional time or cost to be expended during implant, or can result in a need for follow-up corrective action shortly or long after an initial implant procedure.

Accordingly, the present inventors have also recognized that one or more leads can be permanently attached to an implantable pulse generator, such as during manufacturing. In an example, a permanently attached lead can be formed integrally as a portion of a header, such as permanently attached to such a header, and can allow such a header to be more compact, both linearly and volumetrically, as compared to a header including a user-attachable lead coupling. In an example, a header including a permanently attached lead can omit set screws and many of the seals that would generally be included as a portion of a header having a user-attachable lead. Such mechanical simplification can both reduce an error-likelihood during implantation, and can enhance reliability by reducing a size or complexity of the header design, or the complexity of the implantation procedure.

In an example, a subcutaneously-implantable medical device can include a header assembly, an intravascularly-deliverable lead assembly, and a first housing comprising a pulse generator circuit. The header assembly can include a stylet access port. The intravascularly-deliverable lead assembly can be permanently attached to the header assembly and can include a lumen that can receive a stylet via the stylet access port included in the header assembly. The intravascularly-deliverable lead assembly can also include at least one electrode.

In an example, the first housing can include a pulse generator circuit. The first housing can be mechanically coupled to the header assembly, and the pulse generator circuit can be electrically coupled via the header assembly to at least one electrode included as a portion of the intravascularly-deliverable lead assembly. In an example, the header assembly can include a user-accessible electrical contact conductively coupled to at least one electrode included as a portion of the intravascularly-deliverable lead assembly.

In an example, a technique, such as a method, can include forming a header assembly including a stylet access port, and permanently attaching an intravascularly-deliverable lead assembly to the header assembly. The intravascularly-deliverable lead assembly can include at least one electrode, and a lumen to receive a stylet via the access port included in the header assembly. The technique can include forming a first housing comprising a pulse generator circuit. The technique can include mechanically coupling the first housing to the header assembly and electrically coupling the pulse generator circuit to the at least one electrode included as a portion of the intravascularly-deliverable lead assembly via the header assembly.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A through 1C illustrate generally an example that can include a subcutaneously-implantable medical device.

FIG. 2 illustrates generally an example that can include a header assembly of a subcutaneously-implantable medical device.

FIG. 3 illustrates generally an example that can include a header assembly of a subcutaneously implantable medical device.

FIG. 4 illustrates generally an example that can include engagement of the lead assembly by a stylet, such as a via a stylet access port.

FIG. 5 illustrates generally an example that can include an implantable lead having a strain relief portion.

FIG. 6A illustrates generally an example of an implantable medical device that can include a detachable housing.

FIGS. 6B through 6C illustrate generally examples of detachable housings that can be attached to an implantable medical device.

FIG. 7 illustrates generally an example, such as a method, that can include forming a header assembly of an implantable medical device.

### DETAILED DESCRIPTION

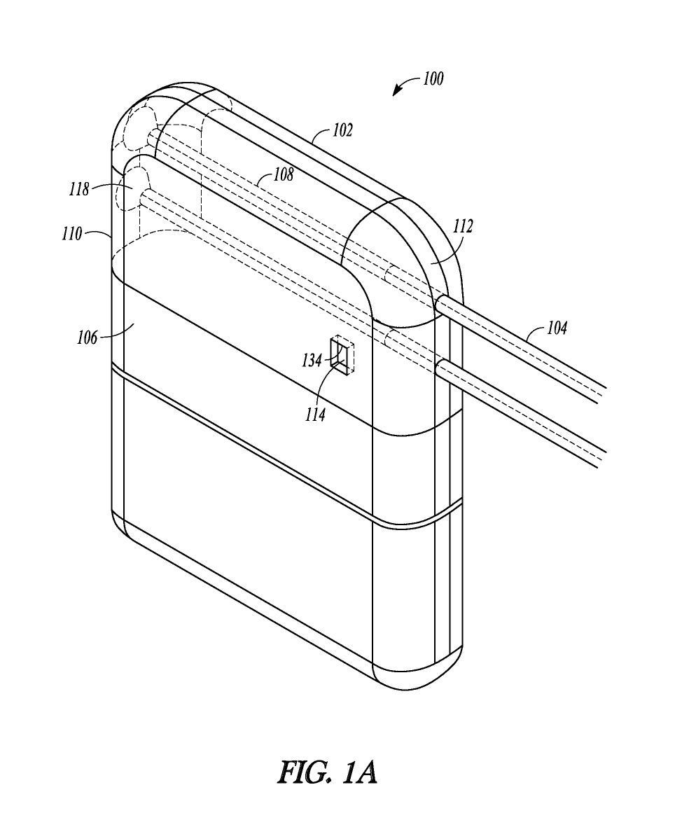

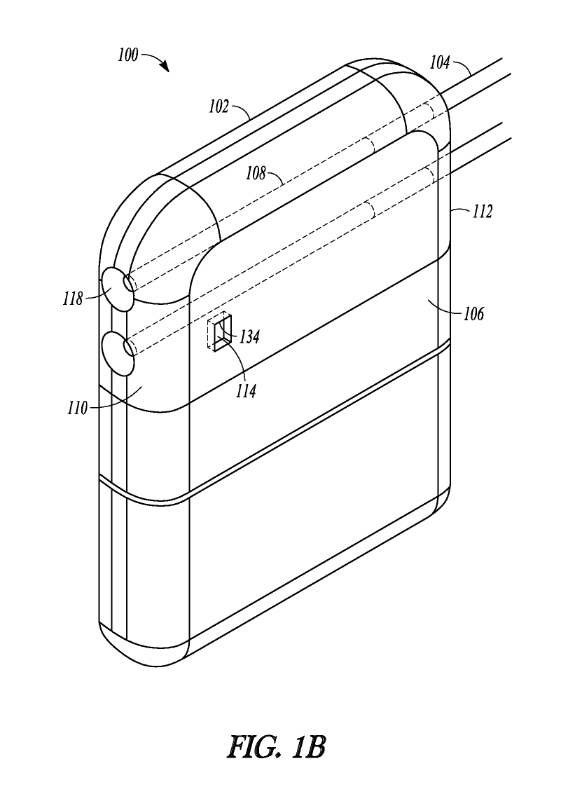

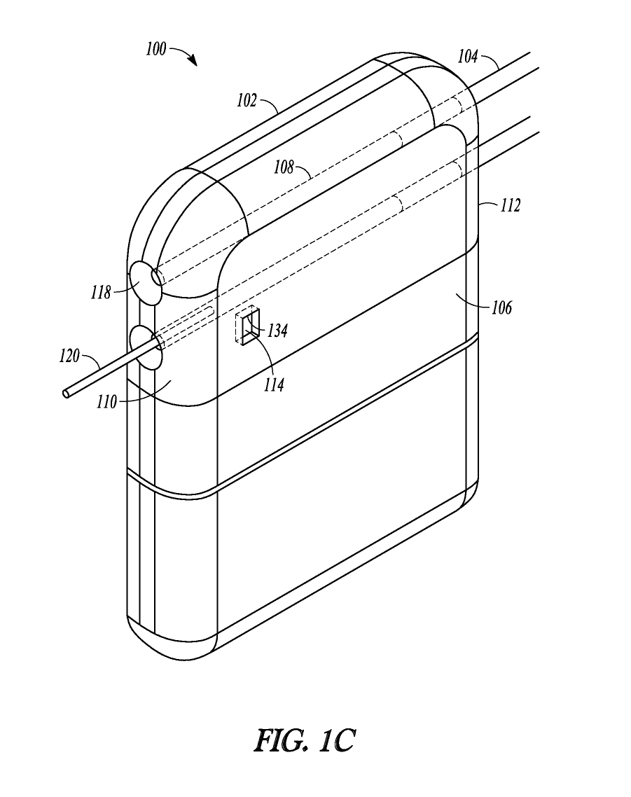

FIGS. 1A through 1C illustrate generally an example that can include a subcutaneously-implantable medical device 100. In an example, the IMD 100 can include a header assembly 102, a first housing 106, and an intravascularly-deliverable lead assembly 104, such as permanently attached to the header assembly 102. For example, the first housing 106 can include a pulse generator circuit that can be electrically and mechanically coupled to one or more implantable leads such as the lead assembly 104, via the header 102. For example, the lead assembly 104 can include one or more electrodes coupled to one or more conductors included in the lead assembly 104, such as mechanically and electrically coupled to a pulse generator circuit located in the first housing 106. In an example, the header assembly 102 can include a stylet access port 118. For example, the intravascularly-deliverable lead assembly 104 can include a lumen that can receive a stylet via the stylet access port 118 included in the header assembly 102.

In an example, the first housing 106, or one or more other portions of the IMD 100 can be hermetically-sealed, such as to isolate internal components within the IMD 100 from the surrounding environment. For example, the IMD 100 can be shaped, sized, or otherwise configured for subcutaneous implant in the body of a patient, or in a sub-muscular location in the body of the patient. In an example, the lead assembly 104 can be implanted in the vasculature of the body of the patient.

The header assembly 102, the lead assembly 104, and the first housing 106 can be connected mechanically and electrically, to generate, deliver, or receive electrical or other signals. In an example, the IMD 100 can perform functions such as physiologic (e.g., cardiac) sensing, arrhythmia or disease diagnosis or status monitoring, cardiac or neurostimulation therapy delivery (e.g, cardiac pacing, defibrillation, cardiac resynchronization therapy, or one or more other therapies), or communication with one or more other implantable or external devices.

In an example, the header assembly 102 of the IMD 100 can include a user-accessible electrical contact 114, a channel 108 coupled to the stylet access port 118, a first face 110, and one or other faces such as a second face 112 substantially opposite to the first face 110. In an example, the header portion can be made up of rigid polymer material. The rigid polymer material can include a medical grade polymer, such as a thermoplastic polyurethane. One or more portions of the lead assembly 104 can include a flexible medical grade polymer such as silicone. A transition portion between the lead assembly 104 and the header assembly 102 can include a material or configuration having an intermediate level of flexibility, such as a thicker cross section or other specified profile, one or more ribbed portions, or including one or more other materials, such as to provide a strain relief to avoid fracturing or damaging the lead assembly 104 at or near its interface with the header assembly 102.

In an example, the user-accessible electrical contact 114 can be conductively coupled to at least a portion of the lead assembly 104, such as via a stamped conductor, etched conductor, or wire that can be welded, or otherwise electrically bonded to a conductor included in the lead assembly 104. For example, the user-accessible electrical contact 114 can be used to provide a user with a temporary electrical connection with the lead assembly 104 during implantation of the IMD 100 such as for determination of pacing parameters, including threshold testing, impedance measurement, or for determination of one or more other parameters.

In an example, an external device, such as an external pacing system analyzer (PSA), can be electrically coupled to one or more user-accessible electrical contacts, such as the electrical contact 114. For example, the user-accessible electrical contact 114 can be used to determine or monitor one or more pacing, sensing, or lead-related parameters before or during implantation of the IMD 100.

In an example, an electrical connector pin or socket, such as included as a portion of a cable assembly attachable to the external device, can be mated with the user-accessible electrical contact 114. For example, one or more connector pins or sockets of such a cable assembly can be mated with respective electrical contacts on or within the header assembly 102, such including the electrical contact 114, to establish a temporary electrical connection with one or more electrodes included as a portion of the lead assembly 104. In an example, the user-accessible electrical contact 114 can be located on the first face 110, second face 112, or elsewhere on the surface of the header assembly 102, or the first housing 106.

In an example, the user-accessible electrical contact 114 can be recessed and can have an electrical contact seal portion 134. The electrical contact seal portion 134 can be used to isolate the user-accessible electrical contact 114 from the fluid around the header assembly 102 before or after the implantation. In an example, such a seal portion 134 can be made of silicone or one or more other flexible polymers, such as including one or more leaflets configured to allow an externally-inserted pin or socket to pierce or displace the one or more leaflets. In an example, the seal portion 134 can be configured to re-seal upon removal of the inserted pin or socket, to again isolate the electrical contact 114 from the environment surrounding the header assembly 102.

The channel 108 can provide a cavity through the header assembly 102 from the first face 110 to one or more of the second face 112 or at least to a lumen portion of the lead assembly 104, such as illustrated in the example of FIG. 1C. In an example, the channel 108 can have a wall made of a medical grade polymer, such as comprising the same material as the header 102 or comprising a portion of the header 102. In an example, the wall can define a boundary of hollow space of the channel 108 within the header assembly 102. In an example, the header assembly 102 can have more than one channel similar to the channel 108, such as respectively corresponding to one or more lead assemblies.

In an example, the lead assembly 104 can be located (e.g., permanently fixed) within the header nearby the channel 108. For example, the lead assembly 104 can be attached so as to prevent or inhibit user-detachment from the header assembly 102. In an example, permanent fastening techniques can include overmolding a portion of the lead assembly 104 with the header assembly 102, or attaching or overmolding a portion of the lead assembly 104 along with an insert to be included on or within the header assembly 102, ultrasonically or thermally bonding a portion of the lead assembly 104 to the header assembly 102, pressing the lead assembly into the header assembly 102, or using one or more of a snap-fitting or sping-clip apparatus. One or more such techniques or apparatus can be used, or other techniques or apparatus can be used, such as to provide permanent coupling of the lead into the header in a non-detachable manner during manufacturing.

In an example, the lead assembly 104 can be fixed to the header assembly 102 such that transverse motion or horizontal motion of the lead is restricted by the header assembly 102, but the lead assembly 104 can be configured to rotate around a central axis of the lead assembly 104 after attachment to the header assembly 102.

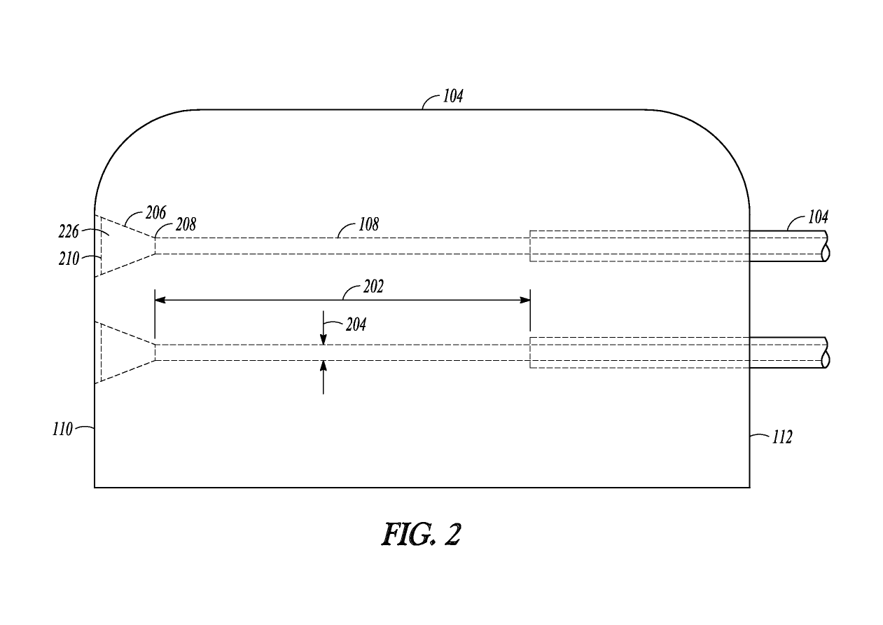

FIG. 2 illustrates generally an example that can include a header assembly of a subcutaneously-implantable medical device, such as the header assembly 102 shown in the example of FIGS. 1A through 1C, or in one or more other examples. A wall of the channel 108 can define a first length 202 such that the first length 202 extends from at or nearby a first face 110 of the header assembly 102 to at least a lumen portion of a permanently-attached implantable lead assembly 104.

In an example, the channel 108 can be defined along a longitudinal direction of the header assembly 102. In an example, the channel 108 can be inclined or bent with respect to the longitudinal direction of the header assembly 102. For example, a direction of the channel 108 through the header assembly 102 need not be collinear with the central axis of the lead or the longitudinal axis of the header assembly 102. In an example, the wall of the channel 108 can define a first diameter or depth 204. In an example, the dimension of the first depth 204 can vary across the first length 202 of the wall. In an example, the dimension of the first depth 204 can be same across the first length 202 of the channel 108.

In an example, the first face 110 can include one or more access ports, such as a stylet access port 226 to allow an implanting caregiver to insert a stylet or apparatus into a lumen of the permanently-attached implantable lead assembly 104 to aid in implanting the lead. For example, the access port 226 can include a shoulder 206, a transition 208, and a seal portion 210.

In an example, the shoulder 206 can define a recess located on the first face 110 of the header assembly 102. The shoulder 206 can be sized, shaped, or configured to guide a specified stylet configuration or design. For example, the shoulder 206 can include a tapered profile such as to guide the stylet into the channel 108. The access port 226 can be sealed from the surrounding environment such as using the seal portion 210. For example, as discussed in one or more examples above in relation to the seal portion for the user-accessible electrical contact, the seal can include a silicone or other elastomeric polymer material, such as comprising one or more leaflets. In an example, a user can pierce or otherwise insert a stylet or other tool through the seal during implantation, such as to manipulate the lead assembly 104. Upon removal of the stylet or other tool, the seal portion 210 can re-seal the access port 226, isolating the channel 208 and interior of the lead assembly 104 from the environment surrounding the header assembly 102.

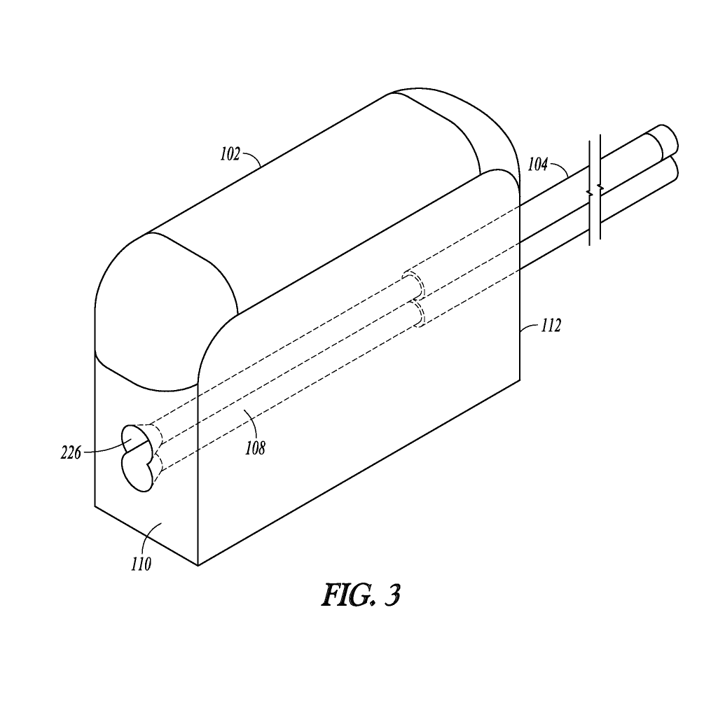

FIG. 3 illustrates generally an example that can include a header assembly 102 of a subcutaneously-implantable medical device. In an example, a channel 108 can include multiple internal passages from a first face 110 of the header assembly 102 to at least a lumen of a lead assembly 104. The internal passages of the channel 108 can be used to guide respective stylets or other tools from one or more access ports, such as an access port 226, to a respective lumen included as a portion of one or more lead assemblies, such as to accommodate a header including multiple permanently-attached lead assemblies.

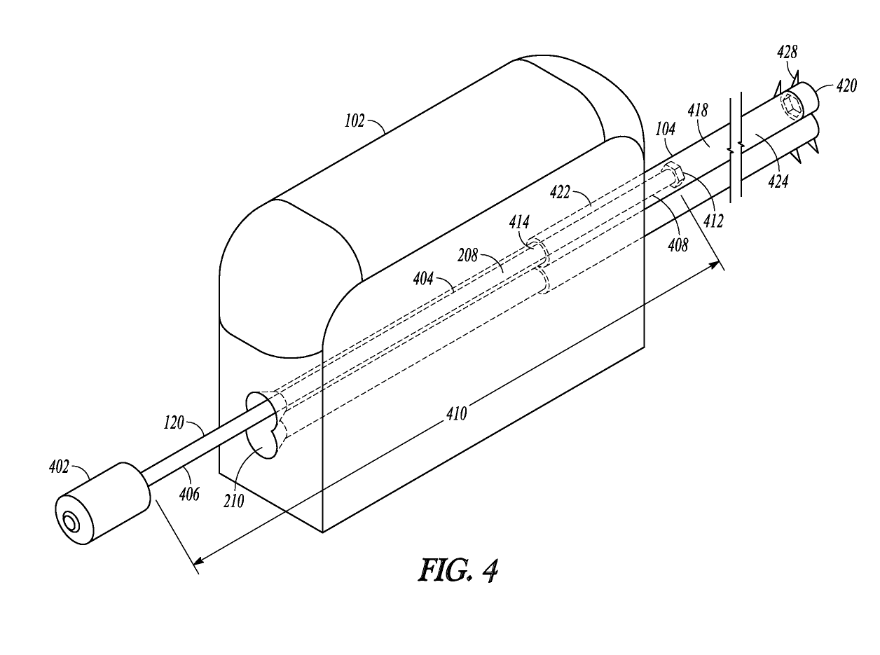

FIG. 4 illustrates generally an example that can include engagement of the lead assembly 104 by a stylet 120, such as a via a stylet access port. In an example, the seal portion 210 of a stylet access port can permit insertion of a tool or stylet. For example, the tool or stylet can be configured for use in manipulating a lead during implantation in the heart (e.g., for guiding the lead, or to help in deploying a fixation device to securely anchor the lead). In an example, a tool 402 (or one or more other tools) can be used to actuate the stylet 120. In an example, the seal portion 210 can be configured to re-seal the interior portion 214 of the header assembly 102 from bodily fluid after the tool 402 or the stylet 120 is removed from the interior portion of the header assembly 102. In an example, the seal portion 210 can be permanently attached to the header assembly 102. For example, the seal portion 210 (or one or more other seal portions) can be fabricated as a “plug” that can be inserted, adhered, or otherwise bonded to a cavity or other location on or within the header assembly 102.

In an example, the stylet 120 can include a body portion 404, a proximal portion 406, and a tip portion 408. In an example, the body portion 404 of the stylet 120 can define a stylet length 410. In an example, the stylet 120 can be used to guide, steer, or stiffen the lead assembly 104 during the implantation of the IMD 100. The stylet 120 can be manipulated using the tool 402. For example, the tool 402 can be permanently or removably coupled to a proximal portion 406 of the stylet 120. In an example, the proximal portion 406 of the stylet 120 can be sized, shaped, or configured to receive the tool 402 such as to couple the tool 402 to the proximal portion 406 of the stylet 120. For example, the stylet 120 may include a hexagonal, Phillips, slotted, or other receptacle configured to mate with a complementary hexagonal, Phillips, slotted or other tip of the tool 402.

In an example, a torque can be applied to one or more of the tool 402 or stylet 120, such as to extend, retract, or otherwise actuate a fixation device at or near a distal tip of the lead assembly 104, such as one or more straight or curled tines, barbs, or helical shapes (e.g., a “cork screw”). For example, the stylet 120 can be used to implant, extract, or explant the lead assembly 104 from the body of a patient. In an example, the stylet 120 can be configured to receive a torque imparted by the tool 402 or configured to deploy the lead assembly 104 at least in part using the torque imparted by the tool 402, or to retract one or more portions of the lead assembly 104 or otherwise assist in explanting the lead assembly 104. Such extraction capability can help to reduce a likelihood that a lead assembly 104 will need to be abandoned in-place rather than explanted in the event of a lead assembly 104 failure, or a failure elsewhere in an IMD 100 that is permanently attached to the lead assembly 104.

In an example, one or more portions of the lead assembly 104 can penetrate into the header assembly 102, such as extending to an internal location 414 to meet the channel 208 from the stylet access port. The lead assembly 104 can be flexible in nature and can be fabricated from a medical grade polymer. In an example, the lead assembly 104 can be silicone, at least in part, and can include a proximal portion 422, a distal portion 424, a lumen 418 extending from the proximal portion 422 to the distal portion 424, an electrode 420, and an user-actuatable fixation device 428. In an example, stylet 120 can be configured to actuate the fixation device 428 such as via a distal tip 412. For example, the distal tip 412 can be sized, shaped, or otherwise configured to engage a portion of the lead assembly 104, such as to apply a torque to the fixation device 428, or generally to the distal portion 424 of the lead assembly 104.

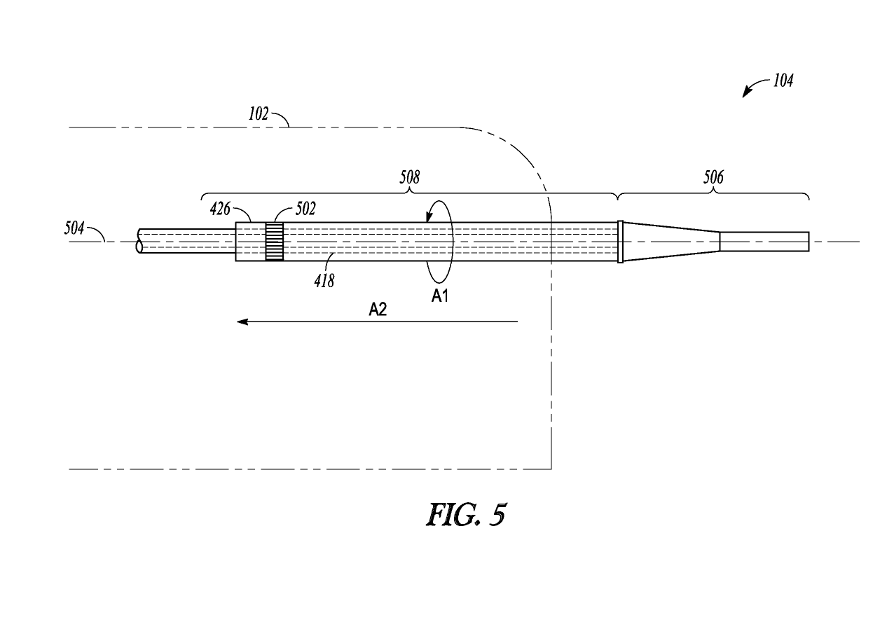

FIG. 5 illustrates generally an example that can include an implantable lead assembly 104 having a sheath 426 comprising a strain relief portion of the lead assembly 104. In an example, the sheath 426 can include a clip or washer portion 502. The clip or washer portion 502 can be configured to allow rotation of the lead assembly 104 around a central axis 504 of the lead assembly 104 along the direction A1 as shown (or rotation in the opposite direction). In an example, the clip or washer portion 502 can be configured to maintain an electrical connection between an internal conductor included as a portion of the rotatable lead assembly 104 and a stationary contact in the header assembly 102 (e.g., a ferrule or other structure configured to surround at least a portion of the circumference of the lead assembly 104), such as during rotation of the lead assembly 104. The rotation of the lead assembly 104 can allow for reduced or more evenly distributed stress on the lead assembly, such as during winding of any excess lead length around the housing or header assembly 102 of the IMD during or after implantation of the device.

In an example, the sheath 426 can include a distal strain relief region 506 (e.g., located in a distal direction from the header assembly 102), such as configured (e.g., sized, shaped, or including materials of intermediate flexibility) such as to provide an intermediate amount of bending flexibility as compared to a more flexible distal portion of the lead assembly 104 and a rigid portion of the header assembly 102.

In an example, the lead assembly 104 can include a proximal strain relief region 508, such as included at least partially within the body of the header assembly 102. One or more of the distal strain relief 506 or the proximal strain relief region 508 can reduce or more evenly distribute stress (e.g., due to bending of the lead assembly toward a direction off the central axis 504), to prevent fatigue, cracking, fracture, or other damage to the lead assembly or its internal conductors.

In an example, one or more of the proximal strain relief region 508 or the distal strain relief region 506 can comprise a portion of the sheath 426, such as including a specified profile or other features (e.g., including a tapered profile, one or more ribs, or one or more other features or materials). In an example, the header assembly 102 can include a rigid polymer material such as thermoplastic polyurethane and the lead assembly 104 can include, at least in part, a flexible material such as silicone.

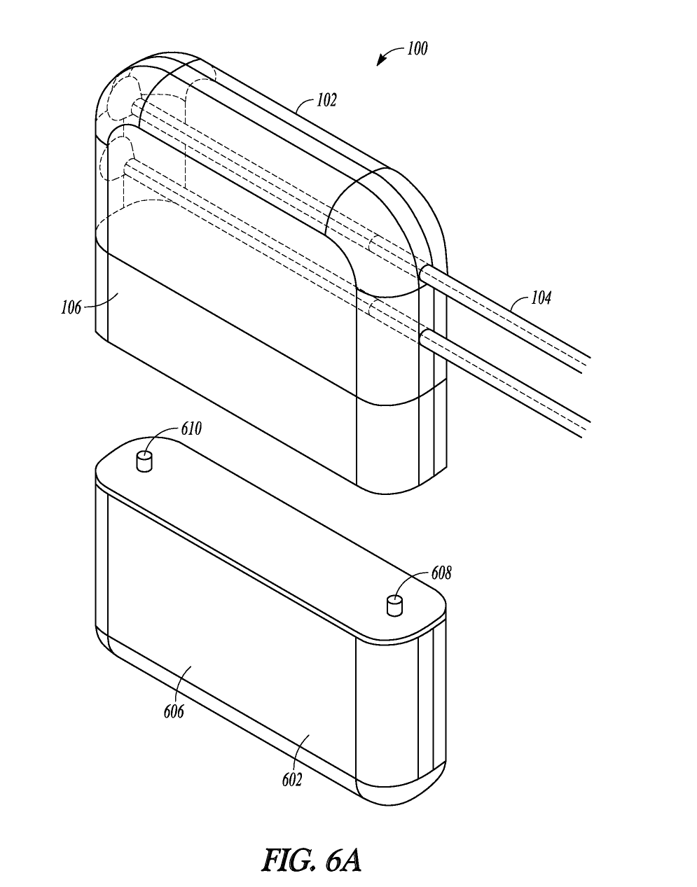

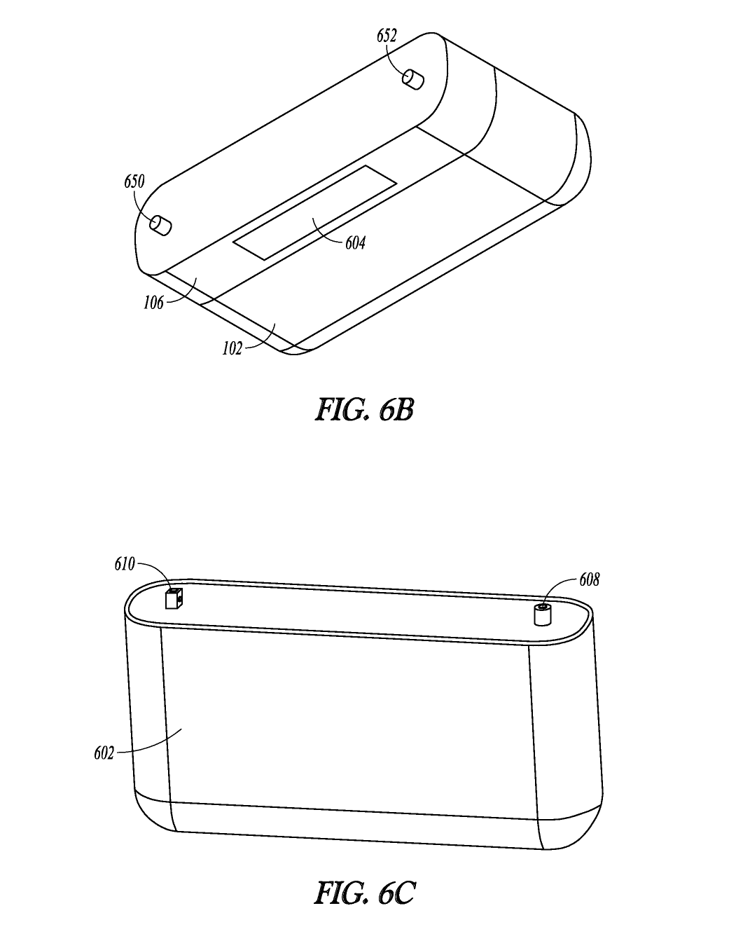

FIG. 6A illustrates generally an example of an implantable medical device 100 that can include a detachable housing. In an example, a first housing 106 can be removably coupled to a second housing 602. In an example, one or more of the first housing 106 or the second housing 602 can be hermetically sealed from the surrounding environment, or from each other.

In an example, the first housing 106 can include a pulse generator circuit 604 (e.g., as shown in FIG. 6B). In an example, the pulse generator circuit 604 can be electrically coupled via the header assembly 102 to an electrode included as a portion of a lead assembly 104. The lead assembly can be permanently attached to the header 102. The pulse generator circuit 604 can include one or more circuits related to control, sensing, operation, storage, therapy delivery, communication, or one or more other functions of the IMD 100, such as including one or more processors, microcontrollers, memories, analog circuits, or digital circuits.

The first housing 106 can include one or more feed-through electrical interconnections, such as a first feedthrough 608 or a second feed-through 610, such as to connect conductive portions of a header assembly to the pulse generator circuit 604 located internally within the hermetically-sealed first housing 106.

FIGS. 6B through 6C illustrate generally examples of a detachable housing that can be attached to an implantable medical device. A power source included in a second housing 602 can be configured to supply power to the IMD 100, or to provide energy storage for the IMD 100 (e.g., including one or more batteries, capacitors, or other circuitry or other devices). In an example, the power source included in the second housing 602 can be coupled to the pulse generator circuit 604 (e.g., shown in FIG. 6A), to provide operating energy for the pulse generator circuit 604. In an example, the power source included in the second housing 602 can be configured to provide power to the pulse generator circuit 604.

In an example, a first housing 106 and the second housing 602 can include respective mating interconnects that can be configured to mate with each other, such as to provide a secure electrical and mechanical connection between the first housing 106 and the second housing 602. In an example, a first interconnect 610 can include a shape or size that is different from a second interconnect 608.

For example, the first interconnect 610 and the second interconnect 608 can be sized, shaped, or located to permit the second housing 602 to mate with the first housing 106 only when the second housing 602 is positioned in a specific orientation. In an example, the first housing 106 and the second housing 602 can be mechanically coupled at least in part using one or more of a conductive or non-conductive band or strap, or using an adhesive backed sheet or tape. For example, a coupling band can be circumferentially located at or near an interface region between respective faces of the first housing 106 and the second housing 602.

In an example, the first housing 106 or the second housing 602 can include respective conductive portions configured to be electrically coupled to each other to provide a common reference conductor when the first housing 106 is mated to the second housing 602.

The present inventors have recognized, among other things, that a user replaceable power source may allow the pulse generator circuit and header assembly to remain implanted for a duration longer than a life-span of a single non-replaceable battery. In an example, the second housing 602 can include other circuitry, such as to provide upgraded features to the IMD when the second housing 602 is replaced with a new housing, such as having upgraded circuitry or programming.

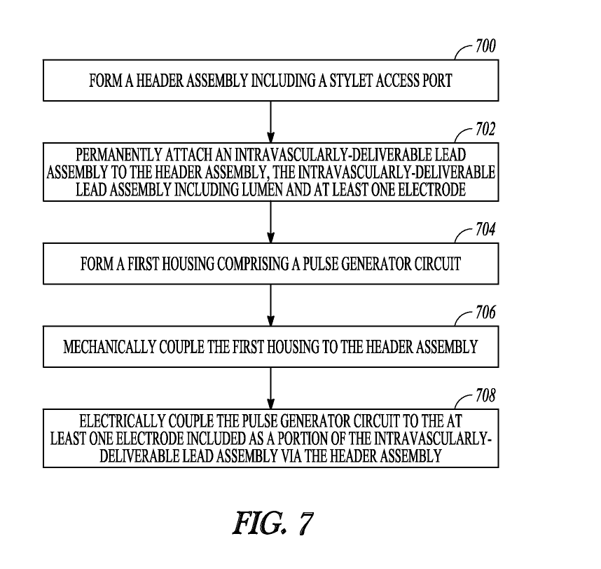

FIG. 7 illustrates generally an example, a method that can include forming a header assembly of an implantable medical device, such as the IMD 100 discussed in one or more examples above. At 700, a header assembly, such as the header assembly 102 discussed in one or more examples above, can be formed to include an access port, such as the stylet access port 226 discussed in one or more examples above. For example, this can include molding (e.g., insert molding), drilling, or otherwise forming a material comprising the header to form an aperture comprising the stylet access port.

At 702, a lead assembly, such as the lead assembly 104 discussed in one or more examples above, can be permanently attached to the header assembly. This can include using a snap-fit technique, an overmolding technique, or one or more other fabrication techniques that can prevent an end user from detaching the lead assembly. In an example, one or more conductive portions of the lead can be welded, soldered, or otherwise bonded to conductors included in the header assembly, such as to mechanically and electrically couple the lead permanently to the header assembly.

At 704, a first housing can be formed, such as the housing 106 discussed in one or more examples above, comprising a pulse generator circuit. At 706, the first housing can be coupled to the header assembly. For example, the first housing can include one or more portions such as one or more posts or anchors, and the header assembly can be glued, molded, welded, or otherwise permanently attached to the first housing.

At 708, the pulse generator circuit can be coupled to at least one electrode included as a portion of the intravascularly-deliverable lead assembly, via the header assembly.

In an example, a technique, such as a method can include forming a user-actuatable fixation device at or near a distal portion of the intravascularly-deliverable lead assembly. In an example, a technique, such as a method, can include forming a user-accessible electrical contact 114 as a portion of the header assembly 102. The user-accessible electrical contact 114 can be conductively coupled to at least one electrode similar to the electrode 420 included as a portion of the intravascularly-deliverable lead assembly 104.

### Various Notes & Examples

Example 1 can include subject matter (such as an apparatus, a method, a means for performing acts, or a machine readable medium including instructions that, when performed by the machine, that can cause the machine to perform acts), such as including a subcutaneously-implantable medical device comprising a header assembly including a stylet access port, an intravascularly-deliverable lead assembly permanently attached to the header assembly and including a lumen to receive a stylet via the stylet access port included in the header assembly and at least one electrode, and a first housing comprising a pulse generator circuit, the first housing mechanically coupled to the header assembly, and the pulse generator circuit electrically coupled via the header assembly to the at least one electrode included as a portion of the intravascularly-deliverable lead assembly.

Example 2 can include, or can optionally be combined with the subject matter of Example 1, to optionally include an intravascularly-deliverable lead assembly comprising a user-actuatable fixation device at or near the distal end of the intravascularly-deliverable lead, the fixation device configured to be actuated by a user via a stylet inserted via the stylet access port included in the header.

Example 3 can include, or can optionally be combined with the subject matter of one or any combination of Examples 1 through 2 to optionally include an intravascularly-deliverable lead extending from a first face of the header assembly, and a stylet access port located on a second face of the header assembly opposite the first face.

Example 4 can include, or can optionally be combined with the subject matter of one or any combination of Examples 1 through 3 to optionally include a header assembly and intravascularly-deliverable lead configured to allow rotation of the lead body around a central axis of the lead.

Example 5 can include, or can optionally be combined with the subject matter of one or any combination of Examples 1 through 4 to optionally include a user-accessible electrical contact conductively coupled to the at least one electrode included as a portion of the intravascularly-deliverable lead assembly.

Example 6 can include, or can optionally be combined with the subject matter of one or any combination of Examples 1 through 5 to optionally include a user-accessible electrical contact comprising a seal portion configured to isolate the user-accessible electrical contact from fluid around the header assembly before or after implantation.

Example 7 can include, or can optionally be combined with the subject matter of one or any combination of Examples 1 through 6 to optionally include a stylet access port comprising a shoulder having a tapered profile configured to guide a stylet, via the stylet access port included in the header, into an interior portion of the header assembly towards the lumen of the permanently-attached intravascularly-deliverable lead.

Example 8 can include, or can optionally be combined with the subject matter of one or any combination of Examples 1 through 7 to optionally include a stylet access port comprising a seal portion configured to isolate an interior portion of the header assembly from a fluid around the header assembly before or after implantation and permit penetration by one or more of a stylet or a tool configured to manipulate the stylet.

Example 9 can include, or can optionally be combined with the subject matter of one or any combination of Examples 1 through 8 to optionally include a seal portion configured to re-seal the interior portion of the header assembly from the fluid when one or more of the stylet or the tool are removed.

Example 10 can include, or can optionally be combined with the subject matter of one or any combination of Examples 1 through 9 to optionally include a header assembly comprising a rigid polymer material, and a seal portion comprising a flexible polymer material.

Example 11 can include, or can optionally be combined with the subject matter of one or any combination of Examples 1 through 10 to optionally include an intravascularly-deliverable lead comprising a strain-relief region configured to provide an intermediate amount of bending flexibility as compared to a remaining, more-flexible distal portion of the lead and a rigid portion of the header assembly.

Example 12 can include, or can optionally be combined with the subject matter of one or any combination of Examples 1 through 11 to optionally include a power source coupled to the pulse generator circuit, the power source included in a second housing, and the second housing removably mechanically and electrically coupled to first housing, the first and second housings hermetically sealed from the around environment, and from each other.

Example 13 can include, or can optionally be combined with the subject matter of one or any combination of Examples 1 through 12 to optionally include first and second housings comprising respective mating electrical and mechanical interconnects configured to mate with each other, the respective mating interconnects one or more of sized, shaped, or located to permit the second housing to be mated to the first housing only when the second housing is positioned in a specified orientation.

Example 14 can include, or can optionally be combined with the subject matter of one or any combination of Examples 1 through 13 to optionally include a stylet.

Example 15 can include, or can optionally be combined with the subject matter of example 14 to optionally include a proximal end of the stylet configured to be mechanically manipulated using a tool.

Example 16 can include, or can optionally be combined with the subject matter of one or any combination of Examples 14 through 15 to optionally include a proximal end of the stylet configured to be torqued by a tool, the stylet configured to deploy a user-actuatable fixation device at least in part using a torque imparted by the tool.

Example 17 can include subject matter (such as an apparatus, a method, a means for performing acts, or a machine readable medium including instructions that, when performed by the machine, that can cause the machine to perform acts), such as including a subcutaneously-implantable medical device including a header assembly including a stylet access port, an intravascularly-deliverable lead assembly permanently attached to the header assembly and including a lumen to receive a stylet via the stylet access port included in the header assembly, at least one electrode, and a first housing comprising a pulse generator circuit, the first housing mechanically coupled to the header assembly, and the pulse generator circuit electrically coupled via the header assembly to the at least one electrode included as a portion of the intravascularly-deliverable lead assembly. In Example 17, the header assembly can optionally include a user-accessible electrical contact conductively coupled to the at least one electrode included as a portion of the intravascularly-deliverable lead assembly.

Example 18 can include, or can optionally be combined with the subject matter of one or any combination of Examples 1-17 to include, subject matter (such as an apparatus, a method, a means for performing acts, or a machine readable medium including instructions that, when performed by the machine, that can cause the machine to perform acts), such as can include forming a header assembly including a stylet access port, permanently attaching an intravascularly-deliverable lead assembly to the header assembly, the intravascularly-deliverable lead assembly including a lumen to receive a stylet via the stylet access port included in the header assembly and at least one electrode, forming a first housing comprising a pulse generator circuit, mechanically coupling the first housing to the header assembly, and electrically coupling the pulse generator circuit to the at least one electrode included as a portion of the intravascularly-deliverable lead assembly via the header assembly.

Example 19 can include, or can optionally be combined with the subject matter of Example 18 to optionally include forming a user-actuatable fixation device at or near the distal end of the intravascularly-deliverable lead, the fixation device configured to be actuated by a user via a stylet inserted via the stylet access port included in the header.

Example 20 can include, or can optionally be combined with the subject matter of one or any combination of Examples 18-19 to optionally include forming a user-accessible electrical contact as a portion of the header, the user-accessible electrical contact conductively coupled to the at least one electrode included as a portion of the intravascularly-deliverable lead assembly.

Example 21 can include, or can optionally be combined with any portion or combination of any portions of any one or more of Examples 1-20 to include, subject matter that can include means for performing any one or more of the functions of Examples 1-20, or a machine-readable medium including instructions that, when performed by a machine, cause the machine to perform any one or more of the functions of Examples 1-20.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus comprising:
a subcutaneously-implantable medical device including:
a header assembly including a stylet access port;
an intravascularly-deliverable lead assembly permanently attached to the header assembly and including:
a lumen to receive a stylet via the stylet access port included in the header assembly; and
at least one electrode; and
a first housing comprising a pulse generator circuit, the first housing mechanically coupled to the header assembly, and the pulse generator circuit electrically coupled via the header assembly to the at least one electrode included as a portion of the intravascularly-deliverable lead assembly.

2. The apparatus of claim 1, wherein the intravascularly-deliverable lead assembly includes a user-actuatable fixation device at or near the distal end of the intravascularly-deliverable lead; and
wherein the fixation device is configured to be actuated by a user via a stylet inserted via the stylet access port included in the header.

3. The apparatus of claim 1, wherein the intravascularly-deliverable lead extends from a first face of the header assembly; and
wherein the stylet access port is located on a second face of the header assembly opposite the first face.

4. The apparatus of claim 1, wherein the header assembly and the intravascularly-deliverable lead are configured to allow rotation of the lead body around a central axis of the lead.

5. The apparatus of claim 1, wherein the header assembly includes a user-accessible electrical contact conductively coupled to the at least one electrode included as a portion of the intravascularly-deliverable lead assembly.

6. The apparatus of claim 5, wherein the user-accessible electrical contact includes a seal portion configured to isolate the user-accessible electrical contact from fluid around the header assembly before or after implantation.

7. The apparatus of claim 1, wherein the stylet access port includes a shoulder having a tapered profile configured to guide a stylet, via the stylet access port included in the header, into an interior portion of the header assembly towards the lumen of the permanently-attached intravascularly-deliverable lead.

8. The apparatus of claim 1, wherein the stylet access port includes a seal portion configured to:
isolate an interior portion of the header assembly from a fluid around the header assembly before or after implantation; and
permit penetration by one or more of a stylet or a tool configured to manipulate the stylet.

9. The apparatus of claim 8, wherein the seal portion is configured to re-seal the interior portion of the header assembly from the fluid when one or more of the stylet or the tool are removed.

10. The apparatus of claim 8, wherein the header assembly comprises a rigid polymer material, and wherein the seal portion comprises a flexible polymer material.

11. The apparatus of claim 1, wherein the intravascularly-deliverable lead includes a strain-relief region configured to provide an intermediate amount of bending flexibility as compared to a remaining, more-flexible distal portion of the lead and a rigid portion of the header assembly.

12. The apparatus of claim 1, comprising a power source coupled to the pulse generator circuit, the power source included in a second housing, and the second housing removably mechanically and electrically coupled to first housing; and
wherein the first and second housing are hermetically sealed from the around environment, and from each other.

13. The apparatus of claim 12, wherein the first and second housings include respective mating electrical and mechanical interconnects configured to mate with each other, and wherein the respective mating interconnects are one or more of sized, shaped, or located to permit the second housing to be mated to the first housing only when the second housing is positioned in a specified orientation.

14. The apparatus of claim 1, further comprising the stylet.

15. The apparatus of claim 14, wherein a proximal end of the stylet is configured to be mechanically manipulated using a tool.

16. The apparatus of claim 15, wherein the proximal end of the stylet is configured to be torqued by the tool; and
wherein the stylet is configured to deploy a user-actuatable fixation device at least in part using a torque imparted by the tool.

17. An apparatus comprising:
a subcutaneously-implantable medical device including:
a header assembly including a stylet access port;
an intravascularly-deliverable lead assembly permanently attached to the header assembly and including:
a lumen to receive a stylet via the stylet access port included in the header assembly; and
at least one electrode; and
a first housing comprising a pulse generator circuit, the first housing mechanically coupled to the header assembly, and the pulse generator circuit electrically coupled via the header assembly to the at least one electrode included as a portion of the intravascularly-deliverable lead assembly; and
wherein the header assembly includes a user-accessible electrical contact conductively coupled to the at least one electrode included as a portion of the intravascularly-deliverable lead assembly.

18. A method, comprising:
forming a header assembly including a stylet access port;
permanently attaching an intravascularly-deliverable lead assembly to the header assembly, the intravascularly-deliverable lead assembly including:
a lumen to receive a stylet via the stylet access port included in the header assembly; and
at least one electrode;
forming a first housing comprising a pulse generator circuit;
mechanically coupling the first housing to the header assembly; and
electrically coupling the pulse generator circuit to the at least one electrode included as a portion of the intravascularly-deliverable lead assembly via the header assembly.

19. The method of claim 18, comprising forming a user-actuatable fixation device at or near the distal end of the intravascularly-deliverable lead, the fixation device configured to be actuated by a user via a stylet inserted via the stylet access port included in the header.

20. The method of claim 18, comprising forming a user-accessible electrical contact as a portion of the header, the user-accessible electrical contact conductively coupled to the at least one electrode included as a portion of the intravascularly-deliverable lead assembly.
